(19) United States
(12) Patent Application Publication
(10) Pub. No.: US 2015/0002331 A1
(43) Pub. Date: Jan. 1, 2015

(54) RADAR SYSTEM FOR MEDICAL USE

(71) Applicant: SIEMENS AKTIENGESELLSCHAFT, Munich (DE)

(72) Inventors: Thomas ALLMENDINGER, Forchheim (DE); Thilo HANNEMANN, Erlangen (DE); Andre HENNING, Erlangen (DE); Javier PENA, Nuernberg (DE)

(73) Assignee: SIEMENS AKTIENGESELLSCHAFT, Munich (DE)

(21) Appl. No.: 14/295,640

(22) Filed: Jun. 4, 2014

(30) Foreign Application Priority Data
Jul. 1, 2013 (DE) 102013212819.3

(51) Int. Cl. G01S 7/02; A61B 5/113

(52) U.S. Cl. CPC G01S 7/02; A61B 5/113
USPC 342/175; 600/595

(57) ABSTRACT

A radar system, a medical diagnostic or therapeutic device and a method are disclosed for operating a radar system. An embodiment of the radar system includes an antenna arrangement embodied to be flat including individually actuatable transmit units for the transmission of radar signals and individually readable receive units for the receipt of radar signals. The transmit units and the receive units each include at least one antenna. Because the transmit units can be individually actuated and the receive units can be individually read out, the information content which can be obtained even without a strong spatial orientation of the radar beam, is increased. According to an embodiment of the invention, the radar system is designed to assign a radar signal received by a receive unit to the transmit unit which transmitted the radar signal received.

### PRIORITY STATEMENT

The present application hereby claims priority under 35 U.S.C. §119 to German patent application number DE 102013212819.3 filed Jul. 1, 2013, the entire contents of which are hereby incorporated herein by reference.

### FIELD

At least one embodiment of the invention generally relates to a radar system, a medical diagnostic or therapeutic device and/or a method for operating a radar system.

### BACKGROUND

For many medical examinations and treatments it is advantageous to record motions of a patient, for example the heartbeat or respiratory motion. In examinations or treatments using imaging modalities such as computed tomography or magnetic resonance tomography it may be important to record the motion of a patient. Furthermore, recording the motion of a patient may also be important for therapeutic treatment using a radiotherapy device. The motion recorded may be used for motion correction of the image data obtained or for triggering. Often the motion data provides information about physiological parameters such as the heart rate or respiratory rate. In order to determine such motions or physiological parameters, the use for example of an ECG to determine the heart rate, and the use of a respiratory belt to determine the respiratory rate, are known. However the need to apply electrodes and/or the breathing belt takes up a certain amount of time, which extends the examination. Moreover, these measures are frequently felt by patients to be unpleasant.

The radar technique is a known technique for contactless detection of objects, their spacing and their motions by emitting electromagnetic signals and receiving the reflected signals. From DE 10 2009 021 232 A1 a patient table for an imaging medical device is known, having a patient positioning plate which has at least one radar antenna. Using the at least one radar antenna, primary signals in the form of electromagnetic waves are emitted in the direction of the patient. If the patient positioning plate in contrast has several radar antennas, each of the radar antennas can emit primary signals in the direction of the patient. These primary signals are reflected by the patient and the organs inside the patient and generate secondary signals. Accordingly these secondary signals can be received by one or more radar antennas and fed to the control and evaluation device. Furthermore, an array of radar antennas is disclosed, in which the correlation of the signals from several antennas can be used to obtain information, in particular to obtain information about the respiration and heartbeat of a patient.

### SUMMARY

At least one embodiment of the invention provides a radar system and/or a method for operating a radar system for medical use, in particular to determine the motion of an examination region of a patient.

A radar system, a medical diagnostic and therapeutic device, and a method are disclosed.

Features, advantages or alternative embodiments mentioned in the process can also be applied and vice versa. In other words, claims which are directed toward a system for example, can also be developed with the features described or claimed in connection with a method. The corresponding functional features of the method are hereby formed by corresponding objective modules.

The inventive radar system of at least one embodiment is provided for medical use. It comprises an antenna arrangement, embodied to be flat, with individually actuatable transmit units for transmitting radar signals and with individually readable receive units for receiving radar signals. The transmit units and the receive units each comprise at least one antenna. Because the transmit units can be individually actuated and the receive units can be individually read out, the information content, in particular the spatial information content, which can be obtained even without a strong spatial orientation of the radar beam, is increased.

At least one embodiment of the invention can also be embodied in the form of a medical diagnostic or therapeutic device, comprising at least one embodiment of an inventive radar system which is designed to use the motion determined by the radar system to control the medical diagnostic or therapeutic unit and/or to postprocess data obtained by the medical diagnostic or therapeutic unit. This type of use increases the quality of the diagnosis or treatment, for example by correcting previously recorded image data or triggering an irradiation system.

Furthermore, at least one embodiment of the invention can be embodied as a method for operating a radar system, comprising the transmission of radar signals in the direction of an examination region of a patient, the receipt of radar signals, the read-out of a receive signal corresponding to the radar signal received, the assignment of the radar signals received to the transmit units which transmitted the radar signals received, by correlating the receive signals with the control signal, and the determination of the motion of an examination region of a patient. The method enables a particularly precise determination of the motion of an examination region of a patient.

### BRIEF DESCRIPTION OF THE DRAWINGS

The invention is described and explained in more detail below with reference to the example embodiments illustrated in the figures, in which:

FIG. 1 shows a plan view of an embodiment of an inventive radar system,

FIG. 2 shows a side view of an embodiment of an inventive antenna arrangement,

FIG. 3 shows the curve of the input reflexion factor for an embodiment of inventive reflexion layers,

FIG. 4 shows an embodiment of an inventive antenna,

FIG. 5 shows a circuit diagram according to a first embodiment of an embodiment of the inventive radar system,

FIG. 6 shows a circuit diagram according to a second embodiment of the inventive radar system,

FIG. 7 shows an embodiment of an inventive computed tomography system, and

FIG. 8 shows a flow chart of an embodiment of the inventive method.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

The inventive radar system of at least one embodiment is provided for medical use. It comprises an antenna arrangement, embodied to be flat, with individually actuatable transmit units for transmitting radar signals and with individually readable receive units for receiving radar signals. The transmit units and the receive units each comprise at least one antenna. Because the transmit units can be individually actuated and the receive units can be individually read out, the information content, in particular the spatial information content, which can be obtained even without a strong spatial orientation of the radar beam, is increased.

According to at least one embodiment of the invention, the radar system is designed to assign a radar signal received by a receive unit to the transmit unit which transmitted the radar signal received. The direct assignment of the radar signal received to a transmit unit also corresponds to a spatial assignment of the radar signal received and thus permits a great deal of relevant information about a patient to be obtained. At least one embodiment of the invention in particular allows the motion of a patient to be determined precisely, as well as contactlessly, fast and reliably.

According to another aspect of at least one embodiment of the invention, the transmit units can be actuated using a control signal, it being possible to read out a receive signal corresponding to the radar signal received, with the radar system being designed to assign by correlating the control signal with the receive signal.

According to another aspect of at least one embodiment of the invention, the radar system comprises a determination unit, designed to record the motion of an examination region of a patient using the correlated receive signals.

According to another aspect of at least one embodiment of the invention, the radar system is designed to transmit radar signals with a particular time offset. Such a time offset is technically simple to achieve and makes possible both a temporally and spatially precise assignment of the radar signal received.

According to another aspect of at least one embodiment of the invention, the radar system is designed to transmit radar signals in a particular sequence of transmit units with a particular scan rate. Depending on the scan rate selected, motions having different frequencies can thereby be recorded. A correspondingly high scan rate for example enables the heart rate to be determined.

According to another aspect of at least one embodiment of the invention, the radar system is designed to operate in continuous wave mode with a fixed transmission frequency for a particular transmit unit and to assign on the basis of the transmission frequency. Alternatively, the radar system is designed to operate in frequency-modulated continuous wave mode with a fixed frequency modulation for a particular transmit unit. By operating the individual transmit units in continuous wave mode the scan rate and thus the temporal resolution can be still further increased.

According to another aspect of at least one embodiment of the invention, the antennas of the transmit units and of the receive units are each embodied in the form of patch antennas. Patch antennas can be easily and cheaply manufactured and in addition are embodied to be particularly flat, meaning they permit a particular flat and compact embodiment of the antenna arrangement.

According to another aspect of at least one embodiment of the invention, the transmit units and the receive units are surrounded by an electrically nonconductive substrate, with the substrate forming a contiguous mat or plate. Thus the antenna arrangement is embodied to be particularly compact and can be positioned particularly simply under or on a patient mounted on the table. Thus the handling of the antenna arrangement is simplified.

At least one embodiment of the invention can also be embodied in the form of a medical diagnostic or therapeutic device, comprising at least one embodiment of an inventive radar system which is designed to use the motion determined by the radar system to control the medical diagnostic or therapeutic unit and/or to postprocess data obtained by the medical diagnostic or therapeutic unit. This type of use increases the quality of the diagnosis or treatment, for example by correcting previously recorded image data or triggering an irradiation system.

Furthermore, at least one embodiment of the invention can be embodied as a method for operating a radar system, comprising the transmission of radar signals in the direction of an examination region of a patient, the receipt of radar signals, the read-out of a receive signal corresponding to the radar signal received, the assignment of the radar signals received to the transmit units which transmitted the radar signals received, by correlating the receive signals with the control signal, and the determination of the motion of an examination region of a patient. The method enables a particularly precise determination of the motion of an examination region of a patient.

According to another aspect of at least one embodiment of the invention, the transmission and receipt of radar signals takes place with a scan rate of at least 10 Hz, so that the antenna arrangement is designed to record the motion of the lungs of the patient.

According to another aspect of at least one embodiment of the invention, the transmission and receipt of radar signals takes place with a scan rate of at least 500 Hz, so that the antenna arrangement is designed to record the motion of the heart of the patient.

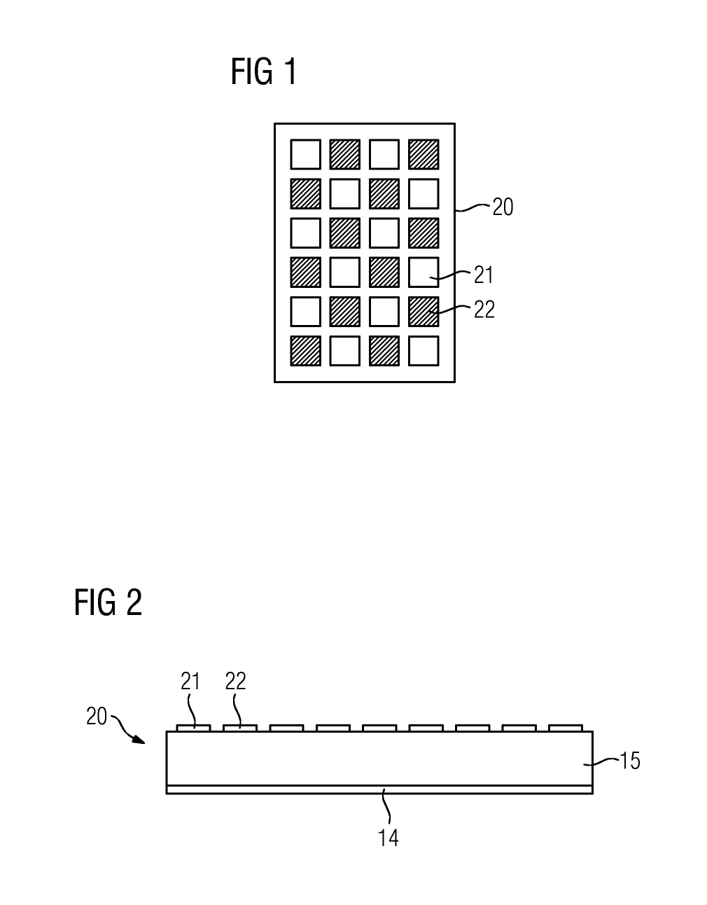

FIG. 1 shows a plan view of an embodiment of an inventive radar system. The radar system comprises an antenna arrangement 20, embodied to be flat, with individually actuatable transmit units 21 for the transmission S of radar signals and with individually readable receive units 22 for the receipt E of radar signals. In the example shown here the transmit units 21 are shown in white and the receive units 22 are hatched. The antennas of the transmit units 21 and of the receive units 22 are each embodied in the form of patch antennas. A patch antenna is a flat, often rectangular antenna, whose edge length can in particular have a value of λ/2, where λ is the wavelength at which the antenna acts as a resonator.

An embodiment of the inventive radar system can be embodied such that both the transmit units 21 and the receive units 22 are designed for the transmission S and receipt E of radar signals. In other words, in certain embodiments of an embodiment of the invention transmit units 21 can act as receive units 22 (and vice versa). An embodiment of the inventive radar system can however also be embodied such that the transmit units 21 are provided only for the transmission S of radar signals and the receive units 22 only for the receipt E of radar signals. In the latter case, the transmit units 21 and the receive units 22 can, as shown here, be arranged in a chessboard pattern; they can however also form other patterns, in so far as this makes technical sense.

Typically the active layer 25 of a patch antenna has a metal. Generally the layer thickness of a metallic active layer 25 is in the order of the skin depth of the metal, which depends on the operating frequency or operating frequencies used. For example, layer thickness of 2 μm to 20 μm are used in the case of metallic active layers 25. The active layer 25 of an embodiment of an inventive antenna arrangement 20 can however also have non-metallic, electrically conductive materials. For example, an active layer 25 for an antenna can have carbon fiber or graphite, since carbon generally absorbs and scatters X-rays 17 less than metals. Antennas with an active layer 25 made of carbon fiber or graphite counter the occurrence of image artifacts if they have to be placed in the beam path of the X-rays 17 during X-ray recordings.

In plan view in the example of the transmit units 20 and receive units 22 shown in FIG. 1, only the antennas are visible in each case. The antennas are embodied identically in the example shown here. The antennas of the transmit units 21 and of the receive units 22 can however also be shaped differently or be otherwise differently embodied, in order to improve the transmit properties or the receive properties. The antennas shown here can have different edge lengths, typically in the region of several centimeters. In particular, resonances at 915 MHz, 868 MHz and 433 MHz are desired, which corresponds to edge lengths of approx. 16.4 cm, 17.3 cm and 34.6 cm in patch antennas. An embodiment of the inventive antenna arrangement 20 visible in FIG. 1 thus typically has dimensions of approx. 0.5 m to 1.5 m wide and 1 m to 2 m long. Both the individual antennas and the entire antenna arrangement 20 can have dimensions and shapes differing from the embodiments cited here by way of example, in so far as this makes technical sense.

FIG. 2 shows a side view of an embodiment of an inventive antenna arrangement. In the example shown here the transmit units 21 and the receive units 22 are applied. In other embodiments (not shown), the transmit units 21 and the receive units 22 may also however not be applied but be completely integrated into the substrate 15. In the present example embodiment of the invention the reflexion layer 14 has an electrically conductive metallic coating. The metallic coating can for example be a coating made of copper which has a thickness of between 2 μm and 20 μm. Alternatively, the reflexion layer 14 can also have a carbon fiber layer, since carbon fiber generally absorbs and scatters X-rays 17 less than metals. The reflexion layer 14 acts as a shield or reflector; in this way a directional effect or directional characteristic is achieved, so that the propagation of the radar signals is essentially limited to that side of the reflexion layer 14 on which the patient 3 is located.

In the example shown here the transmit units 21 and the receive units 22 of the antenna arrangement 20 are surrounded by a nonconductive substrate 15, with the substrate 15 being embodied in the form of a contiguous mat or plate. Depending on the type and processing of the substrate 15 and of the transmit units 21 and receive units 22 the antenna arrangement is therefore embodied as a flexible mat or as a rigid plate. A flexible mat is particularly suitable for being placed on or under a patient 3, in particular on a patient table 6. An antenna arrangement 20 embodied as a solid plate can be embodied as part of a patient table 6 and in particular be integrated therein.

An antenna arrangement embodied as a solid plate need not be embodied to be level, but may also be curved, for example to fit the contour of a patient 3. If the substrate is embodied in the form of a plate, it has a high proportion of FR4 material or Teflon, for example. In contrast, if the substrate is embodied in the form of a flexible mat, it has a high proportion of a porous plastic or of a polyimide, for example. Porous plastic or polyimides are light and absorb X-rays only to a slight extent. There can also be an air layer between the antennas and the reflexion layer 14 of the antenna arrangement 20. The thickness of the entire antenna arrangement 20 in the form of a mat or plate is typically in the region of a few millimeters to a few centimeters.

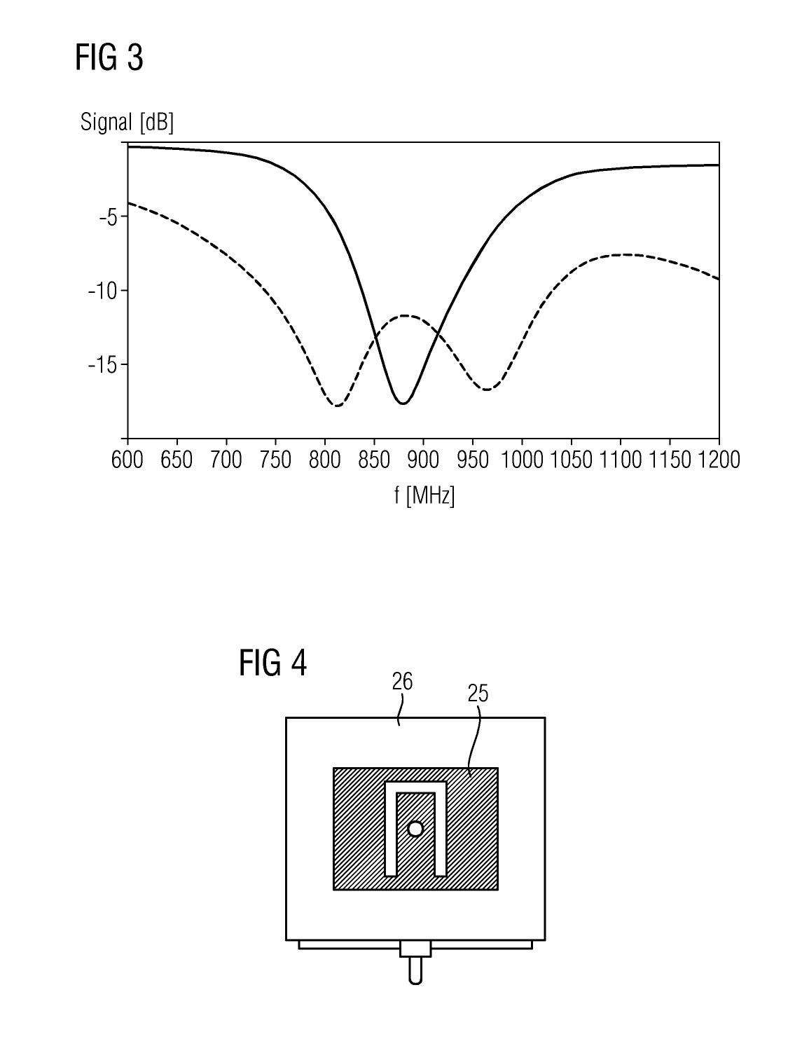

FIG. 3 shows the curve of the input reflexion factor for an embodiment of inventive reflexion layers made of copper or carbon fiber. The dashed line represents the input reflexion factor for an embodiment of an inventive reflexion layer 14 made of carbon fiber, while the solid line represents the input reflexion factor for an embodiment of an inventive reflexion layer 14 made of copper. In this case the S11 coupling between the radar antennas in the form of the reflexion coefficient, designated a “signal” in FIG. 3, is plotted in units of decibels [dB] against the frequency of the radar signal. FIG. 3 shows that the bandwidth of the effectively available radar signal is increased by the use of a reflexion layer 14 made of carbon fiber. A reflexion layer 14 made of graphite has advantageous properties which are similar to a reflexion layer made of carbon fiber.

If during the performance of an embodiment of the inventive method the antenna arrangement 20 is situated in the immediate vicinity of the patient 3, predominantly the near field of the transmitted radar signals is reflected and received by the examination region of the patient 3. Furthermore, the antennas are “mistuned” because of the immediately vicinity of the patient 2, since the dielectric ratios between substrate 15 and the interior of the patient 3 change considerably. Hence a large bandwidth is desirable for a radar system for medical use. If the antenna has only a small bandwidth, there is an increased risk that the transmission frequency will be outside the effective resonance, shifted by the patient 3, of the antenna. If the transmission frequency is outside the effective resonance of the antenna, this results in a smaller amplitude for the receive signal and a low phase shift.

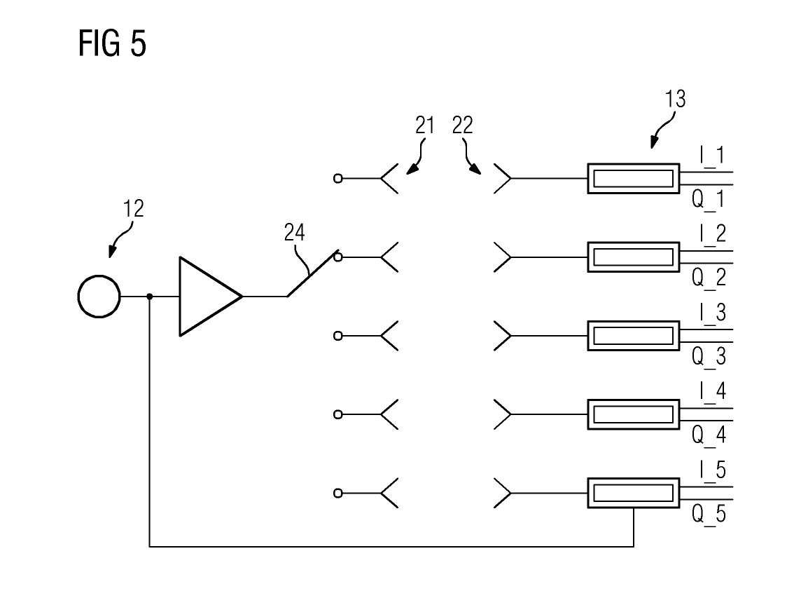

FIG. 4 shows an embodiment of an inventive antenna. The antenna shown here is a patch antenna, with the hatched region representing an active layer 25, for example including a metal, in particular copper, or carbon fiber or graphite. The active layer 25, shown hatched, which exercises the actual function of the antenna, is located on the carrier layer 26 represented in white. This carrier layer typically includes a porous plastic and in the example shown here is embodied to be considerably thicker than the active layer 25. The thickness and the dielectric constant of the carrier layer significantly determine the properties of the antenna. In principle, a greater thickness and/or a greater dielectric constant increases the bandwidth of the antenna.

The “U”-shaped recess in the active layer 25 increases the transmission power or the receive power of the antenna. A connection is shown at the bottom of FIG. 4, via which control signals can be transmitted to the antenna, or via which receive signals from the antenna can be read out. The antenna shown here is particularly suitable for transmitting or receiving radar signals in the frequency range between 100 MHz and 5 GHz. Accordingly, the antenna shown here can in particular be used as part of an embodiment of the inventive radar system or of an inventive medical diagnostic or therapeutic device.

FIG. 5 shows a circuit diagram of an embodiment of the inventive radar system. The local oscillator 12 generates a signal frequency, typically in the range between 100 MHz and 5 GHz. The signal generated by the local oscillator is amplified to the desired transmission power by the power amplifier shown as a triangle. In the example shown here the signal is transmitted by the switch 24 consecutively to the transmit units 21, with each of the transmit units 21 having an antenna for the transmission S of a radar signal with the signal frequency. The radar signals transmitted by a transmit unit 21 can be received by the receive units 22, with each of the receive units 22 comprising an antenna in the example shown here. The receive signals are demodulated by the I/Q demodulators 13 and in each case are converted into an I component (I_{—}1 to I_{—}5) and in each case into a Q component (Q_{—}1 to Q_{—}5). In this case a receive signal is split such that a part is demodulated with the original phase position and produces the I component, with the second part being demodulated phase-shifted by 90° and producing the Q component.

In the example shown here the I/Q demodulator 13 is operated with the same signal frequency as the transmit units 21. In another embodiment, not shown here, the I/Q demodulators 13 are operated with an intermediate frequency which differs slightly, typically in the region of a few kHz, from the signal frequency. Furthermore, the number of transmit units 21 and receive units 22 used can of course vary, in particular the number of transmit units 21 and of receive units 22 in an inventive radar system can differ. Other electronic components such as mixers, filters, amplifiers, etc. can also be used to generate the desired control signal or to demodulate and further process the receive signal, in particular to enable an inventive assignment Z. In another embodiment, the demodulation takes place digitally.

In the embodiment shown here the transmit units 21 do not transmit their respective radar signals simultaneously. Instead the transmit units 21 transmit a temporal series of radar signals, with the transmit units 21 being located at different spatial positions. Thus the transmit units 21 transmit a temporal series which uses the instant of transmission (or receipt) to enable conclusions to be drawn about the spatial position of the transmit unit 21 which transmitted the respective radar signal. However, because of the very small time delay when a radar signal is reflected by a patient 3, the absolute instant of the transmission S of a radar signal is not compared to the receipt E of the radar signal. Instead, conclusions are drawn about the spatial position of the transmit unit 21 which transmitted the radar signal received by correlating the control signal which corresponds to the radar signal transmitted with the receive signal which corresponds to the radar signal received.

It is known in principle from the field of radar technology to draw conclusions about the motion and/or position of an object by correlating a control signal and a receive signal, in particular with the help of an I/Q demodulator. However, it is not known for the information content obtained for medical use using a radar system to be increased by correlating control signals and receive signals. This is particularly the case because the I/Q demodulation can be carried out not only for a permanently assigned pair of antennas, but in principle for the combination of each transmit unit 21 with each receive unit 22. In the embodiment shown here all transmit units 22 can simultaneously receive the radar signals transmitted by a transmit unit 21.

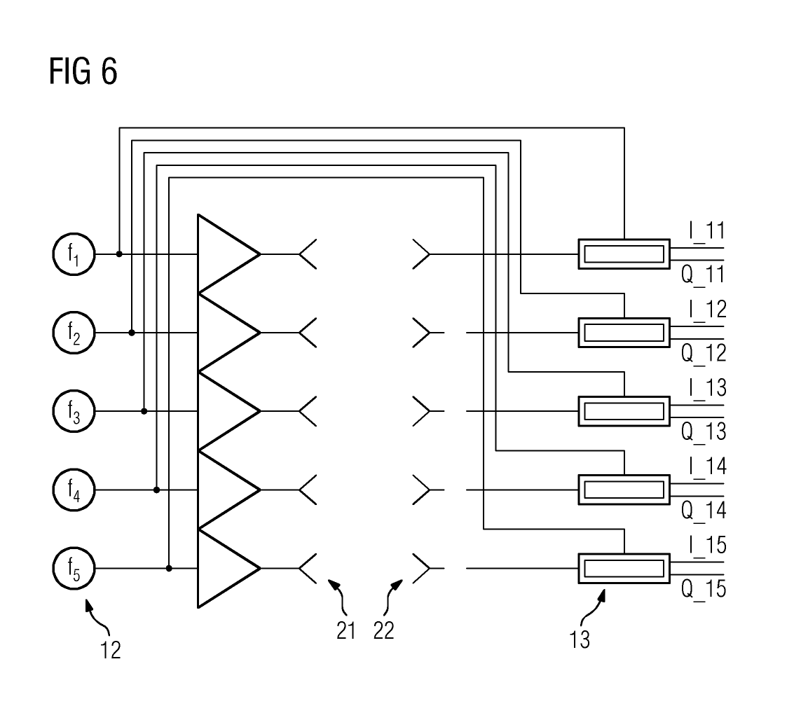

FIG. 6 shows an alternative circuit diagram of an embodiment of the inventive radar system. In the embodiment shown here five transmit units 21 are each operated with different signal frequencies f1 to f5, generated by different local oscillators 12. The signal generated by the local oscillators is amplified to the desired transmission power by the power amplifiers shown as a triangle. In the embodiment shown here five I/Q demodulators 13 are assigned to a receive unit 22 in each case. It is not explicitly shown here that five I/Q demodulators 13 are also assigned in each case to the other four receive units 22. The five I/Q demodulators 13 per receive unit 22 in each case are operated using the signal frequencies f1 to f5. For each of the receive signals, based on the frequencies f1 to f5, a separate I/Q demodulator 13 is therefore present. For all receive units 22 this would be a total of 25 I/Q demodulators 13 in the example shown here. Together these generate 25 I components I_{—}11 to I_{—}55 and 25 Q components Q_{—}11 to Q_{—}55.

The embodiment shown here is particularly suitable for continuous wave operation. Thus the signal frequencies f1 to f5 can each have a fixed, but in each case different, value. It is advantageous here if the differences in the signal frequencies f1 to f5 remain small enough that the antennas do not need to be adjusted differently, for example the frequencies can differ by 1 kHz to 100 kHz in each case. The signal frequencies f1 to f5 can also vary over time and effect a different frequency modulation. According to an embodiment of the invention it is possible in both cases for a radar signal received by a receive unit 22 to be assigned to the transmit unit 21 which transmitted the radar signal received. In other embodiments the signal frequencies can differ considerably, such that the antennas of the transmit units 21 have different dimensions, so that the antennas permit a resonant oscillation at the signal frequency allocated to them in each case.

In the case of a radar system used in continuous wave mode, the complex time-dependent transmission factor can be determined for each evaluated pair of transmit units 21 and receive units 22 in the form of the (real) I and Q component of the receive signal relative to the transmitted radar signal, as a function of the time t: I(t,j), Q(t,j) where j=1 . . . and N is the number of the pairs of antennas evaluated. For other radar modes another type of signal is produced if appropriate, but generally the signal of each antenna pairing can be described as a vector U(t,j) where j=1 . . . N. The variable t may be time-continuous or else time-discrete. In the case of simple continuous wave radar, U would be a two-component vector with the elements I and Q. In the case of multifrequency continuous wave radar, U would contain the I and Q components for each signal frequency, and thus at M signal frequencies would have 2×M components. In the case of ultra-wideband radar the elements of U would correspond to different delays (and thus intervals) between the transmitted radar signal and the received radar signal. The values of U would then describe the correlation between the transmitted radar signal and the received radar signal in the case of the respective delay.

The complexity of the circuit can be reduced in alternative embodiments, by not assigning a separate I/Q demodulator 13 to each receive unit 22 for each transmit unit 21 (or each signal frequency). This may be expedient, since more remote antennas contribute less information on the motion to be determined. In another example intermediate frequencies can be used in each case to operate the I/Q demodulators 13. In another embodiment the demodulation takes place digitally, which is advantageous in that the electronics for digitizing the receive signal received need only be present once per receive unit 22, and in that the plurality of demodulators per receive unit 22 can be fully implemented in software.

A combination of the embodiments shown here is also conceivable, in which switching takes place between different transmit units 21 and a number of transmit units 21 are operated simultaneously with different signal frequencies. In other words, some transmit units 21 can be operated in pulsed mode, while other transmit units 21 are operated in continuous wave mode. Furthermore, it is in principle possible to combine the different embodiments cited here with one another.

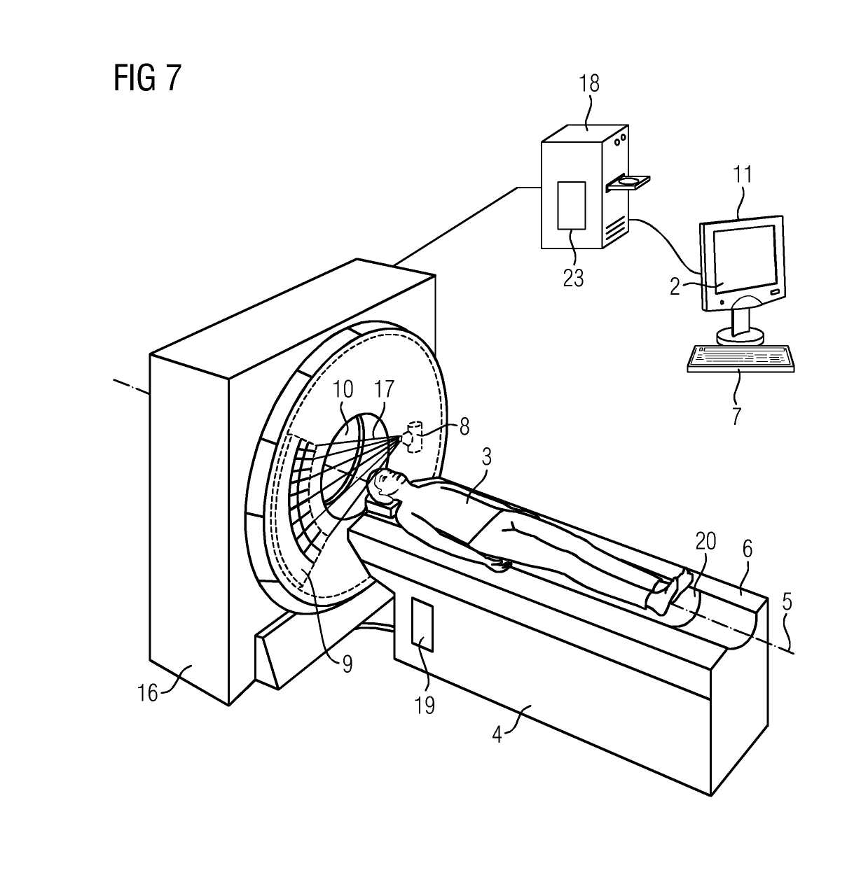

FIG. 7 shows an embodiment of an inventive computed tomography system. The computed tomography system relates to an exemplary embodiment of a medical diagnostic or therapeutic device. The computed tomography system shown here has a recording unit, comprising an X-ray source 8 and an X-ray detector 9. The recording unit rotates about a longitudinal axis 5 during the recording of a tomographic image, and the X-ray source 8 emits X-rays 17 during the spiral recording. While an image is being recorded the patient 3 lies on a patient table 6. The patient table 6 is connected to a table base 4 such that it supports the patient table 6 bearing the patient 3. The patient table 6 is designed to move the patient 3 along a recording direction through the opening 10 of the gantry 16 of the computed tomography system. In the example shown here the antenna arrangement 20 of the inventive radar system is integrated into the patient table 6.

In the present example embodiment the invention comprises a control and evaluation unit 19 which is integrated into the table base 4 and accordingly is always located outside the beam path of the X-rays 17. The control and evaluation unit 19 can additionally, in a manner not shown, be shielded against scattered X-rays, for example with a plate or a housing made of lead. The control and evaluation unit 19 is also connected to the computer 18 to exchange data. The control and evaluation unit 19 can in particular comprise one or more local oscillators 12 and one or more I/Q demodulators 13. In particular, if the antenna arrangement 20 is embodied as a flexible mat which can be placed on the patient 3, the control and evaluation unit 19 can also be accommodated in a separate housing outside the patient table 6 or the table base 4. In each case it is advantageous to protect the control and evaluation unit 19 against X-rays by a corresponding sheathing.

It is the function of the control and evaluation unit 19 to actuate the antenna arrangement 20 and thus the individual transmit units 21 using a control signal and to read out receive signals from the individual receive units 22. The control signal can in particular be generated by at least one local oscillator 12 and if appropriate by further electronic components such as a mixer, amplifier or filter. The control and evaluation unit 19 shown here is designed for the assignment Z of a radar signal received by a receive unit 22 to the transmit unit 21 which transmitted the radar signal received, by correlating the control signal with the receive signal. The control and evaluation unit 19 is furthermore designed to receive signals from a computer 18 or to transmit signals to the computer 18.

In the example shown here the medical diagnostic or therapeutic unit is designed in the form of a computed tomography system by a determination unit 23 in the form of a stored computer program that can be executed on a computer 18, for the determination B of the motion of an examination region of a patient 3. It is generally the case that the determination unit 23 can be embodied in the form of both hardware and software. For example, the determination unit 23 can be embodied as a so-called FPGA (acronym for “Field Programmable Gate Array”) or can comprise an arithmetic logic unit. Other than shown here, the determination unit 23 can also be located in the immediate vicinity of the control and evaluation unit 19 or can be embodied together therewith as a compact unit. In particular the determination unit 23 can also be integrated into the table base 4.

Furthermore, in the example shown here the medical diagnostic or therapeutic unit is designed to use the motion determined by an embodiment of the inventive radar system for the control St of the medical diagnostic or therapeutic unit and/or for the postprocessing Nb of data obtained by the medical diagnostic or therapeutic unit. The data can for example be image data. The medical diagnostic or therapeutic unit can be designed for the control St and the postprocessing Nb in particular by a computer program retrievably stored on the computer 18. The control St comprises the irradiation of the patient 3, for example with electromagnetic radiation, electrons or particles, depending on the type of the medical diagnostic or therapeutic unit. Thus the irradiation may for example take place only in the resting phase of the heart of the patient 3 or a particular position of the thorax of the patient 3 which depends on the respiratory motion. The intensity of the radiation or the angle of radiation can also be adjusted by control St. In another embodiment the control St comprises positioning the patient 3 by moving the patient table 6. The postprocessing Nb relates for example to the segmentation or registration of a temporal series of images, based on image data, of a moving examination region.

The computer 18 is connected to an output unit 11 and an input unit 7. The output unit 11 is for example one (or more) LCD, plasma or OLED screen(s). The output 2 on the output unit 11 comprises for example a graphical user interface for actuating the individual units of the computed tomography system and the control and actuation unit 19. Furthermore, different views of the recorded data can be displayed on the output unit 7. The input unit 7 is for example a keyboard, mouse, so-called touch screen or even a microphone for speech input.

In other example embodiments, not shown here, the medical diagnostic or therapeutic device may relate to imaging devices other than a computed tomography system, for example a magnetic resonance tomography system or a C-arm X-ray device. The medical diagnostic or therapeutic device may furthermore be designed to use positron emission tomography. Furthermore, the medical diagnostic or therapeutic device may relate to a device which is designed to emit electromagnetic radiation and/or electrons and/or particles such as ions for example and thus is suitable for use in radiotherapy or particle therapy.

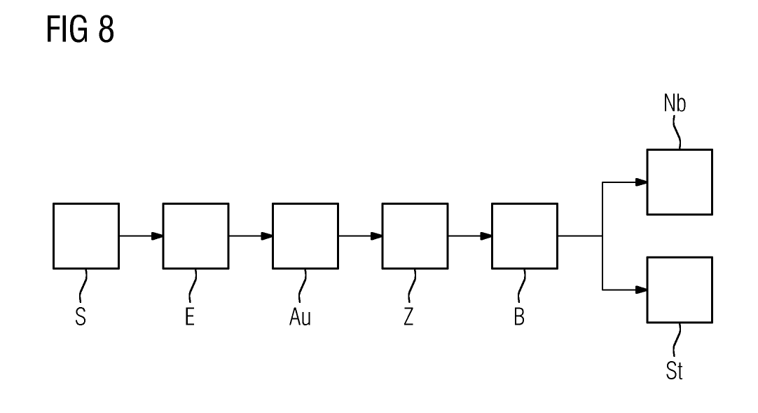

FIG. 8 shows a flow chart of an embodiment of the inventive method for operating a radar system. The inventive method comprises the transmission S of radar signals in the direction of an examination region of a patient 3, the receipt E of radar signals, and the read-out Au of receive signals corresponding to the radar signals received. Furthermore, an embodiment of the inventive method comprises the assignment Z of the radar signals received by the receive units 22 to the transmit units 21 which transmitted the radar signals received in each case. The assignment Z can take place by correlation of the receive signal with the control signals. The direct assignment Z of a received radar signal to a transmit unit 21 also corresponds to a spatial assignment of the received radar signal.

An embodiment of the inventive method also comprises the determination B of the motion of an examination region of a patient 3. Using an embodiment of the inventive method the speed and direction of the motion of the examination region can be determined by way of the Doppler effect from a radar signal transmitted by a transmit unit 21, reflected by the examination region and then received by a receive unit 22. The determination B takes place for example using the determination unit 23. In this way, additionally or alternatively to the direct evaluation on the basis of the Doppler effect, a temporal series of digitized values of the I and Q components obtained from an I/Q demodulator 13 can be adjusted to retrievably stored temporal series of I and Q components which correspond to known motions of the examination region. An embodiment of the invention also allows the motion of a patient 3 to be determined precisely, as well as contactlessly, fast and reliably.

In another embodiment of the invention, the transmission S and receipt E of radar signals takes place with a scan rate of at least 10 Hz, so that the motion of the lungs of the patient 3 can be recorded. In another embodiment of the invention, the transmission S and receipt E of radar signals takes place with a scan rate of at least 500 Hz, so that the motion of the heart of the patient 3 can be recorded. In both these embodiments the radar signals transmitted from the different transmit units 21 must of course be distinguished, for example using a different frequency, a different frequency modulation or a different transmit instant. If the inventive antenna arrangement 20 comprises ten transmit units 21, each with an antenna, and if a scan rate of 10 Hz (or 500 Hz) is aimed for, each of the ten antennas transmits ten radar signals (or 500 radar signals) a second. The scan rate within the meaning of the present application is thus in principle independent of the number of transmit units 21.

For example, all transmit units 21 can transmit a radar signal simultaneously, each with a different frequency, in order to achieve the corresponding scan rate. Operation in continuous wave mode is then possible, so that the scan rate can be very high. Alternatively the transmit units 21 transmit radar signals one after the other, if appropriate with the same frequency. Operation is then in pulsed mode. In particular, the transmit units 21 can transmit radar signals in a fixed sequence in each cycle—i.e. the period in which each antenna transmits exactly one radar signal in pulsed operation—and which lasts a tenth of a second at a scan rate of 10 Hz. In another embodiment, the inventive method is carried out in ultra-wideband mode.

In another embodiment, the inventive method also comprises the control St of a medical diagnostic or therapeutic unit and/or the postprocessing Nb of data obtained by a medical diagnostic or therapeutic unit, in each case using the determined motion of the examination region of the patient 3. An inventive method embodied in this way increases the quality of the diagnosis or treatment, for example by correcting previously recorded image data or triggering an irradiation system.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Although the invention has been illustrated and described in greater detail on the basis of the preferred example embodiments, the invention is not limited by the disclosed examples and other variations can be derived herefrom by the person skilled in the art without departing from the scope of protection of the invention. In particular method steps can be performed in a different sequence from the sequences cited.

What is claimed is:

1. A radar system for medical use, comprising:
an antenna arrangement embodied to be flat, the antenna arrangement including
individually actuatable transmit units for transmission of radar signals, and
individually readable receive units for receipt of radar signals, the transmit units and the receive units each including at least one antenna, wherein the radar system is designed for assignment of the respective radar signals received to the respective transmit units which transmitted the radar signals received.

2. The radar system of claim 1, wherein the transmit units are actuatable using a control signal, wherein receive signals corresponding to the radar signals received can be read out, and wherein the radar system is designed for the assignment by correlating the receive signals with the control signal.

3. The radar system of claim 2, further comprising:
a determination unit, designed for determination of motion of an examination region of a patient using the correlated receive signals.

4. The radar system of claim 1, wherein the radar system is designed for transmission of radar signals with a time offset.

5. The radar system of claim 4, wherein the radar system is designed for transmission of respective radar signals in a sequence from transmit units with a respective scan rate.

6. The radar system of claim 1, wherein the radar system is designed to operate in continuous wave mode with a fixed transmission frequency for a respective transmit unit and for assignment on a basis of a respective transmission frequency.

7. The radar system of claim 1, wherein the radar system is designed to operate in frequency-modulated continuous wave mode with a frequency modulation fixed for a respective transmit unit.

8. The radar system of claim 1, wherein the antennas of the transmit units and of the receive units are each embodied in the form of patch antennas.

9. The radar system of claim 1, wherein the receive units and the transmit units are surrounded by an electrically nonconductive substrate, and wherein the substrate forms a contiguous mat or plate.

10. A medical diagnostic or therapeutic device comprising:
the radar system of claim 3, wherein the medical diagnostic or therapeutic device is designed to use the motion determined by the radar system for at least one of
control of the medical diagnostic or therapeutic unit and
postprocessing of data obtained by the medical diagnostic or therapeutic unit.

11. A method for operating the radar system of claim 3, comprising:
transmitting radar signals in a direction of an examination region of a patient;
receiving radar signals reflected by the examination region;
reading-out receive signals corresponding to the radar signals received; and
assigning the respective radar signals received to respective the transmit units which transmitted the radar signals received, by correlating the receive signals to the control signal.

12. The method of claim 11, further comprising:
determining the motion of an examination region of a patient using the correlated receive signals.

13. The method of claim 12, wherein the transmission and receipt of radar signals takes place with a scan rate of at least 10 Hz, so that the antenna arrangement is designed to record the motion of lungs of the patient.

14. The method of claim 12, wherein the transmission and receipt of radar signals takes place with a scan rate of at least 500 Hz, so that the antenna arrangement is designed to record the motion of a heart of the patient.

15. The radar system of claim 2, wherein the radar system is designed for transmission of radar signals with a time offset.

16. The radar system of claim 3, wherein the radar system is designed for transmission of radar signals with a time offset.

17. A method for operating a radar system, comprising:
transmitting radar signals in a direction of an examination region of a patient;
receiving radar signals reflected by the examination region;
reading-out receive signals corresponding to the radar signals received; and
assigning the respective radar signals received to respective the transmit units which transmitted the radar signals received, by correlating the receive signals to a control signal.

18. The method of claim 17, further comprising:
determining motion of an examination region of a patient using the correlated receive signals.

19. A computer readable medium including program code segments for, when executed on a control device of a radar system, causing the control device of the radar system to implement the method of claim 1.

20. A computer readable medium including program code segments for, when executed on a control device of a radar system, causing the control device of the radar system to implement the method of claim 17.
